(19) United States
(12) Patent Application Publication
(10) Pub. No.: US 2025/0152871 A1
(43) Pub. Date: May 15, 2025

(54) Method and Apparatuses for Maximizing Delivery of Charged Particles to Alveoli for Rapid Absorption

(71) Applicant: Steven Mark Hayden, Reno, NV (US)

(72) Inventor: Steven Mark Hayden, Reno, NV (US)

(21) Appl. No.: 18/509,102

(22) Filed: Nov. 14, 2023

(51) Int. Cl. A61M 15/02; A61M 15/00; G01F 1/56; H02N 1/06

(52) U.S. Cl. CPC A61M 15/02; A61M 15/009; G01F 1/56; H02N 1/06

(57) ABSTRACT

A system and method for delivering charged particles. The system comprises components attached to a patient to effectively deliver aerosolized particles to the patient. Specifically, the system includes a conductive surface, a nebulizer, an insulated chair, and a charging machine. The charging machine is configured to provide an electrical charge to the patient by way of the conductive surface and to the nebulizer. The nebulizer is configured to aerosolize fluid and provide a flow of aerosolized fluid to the patient. The insulated chair is configured to insulate the patient from the ground. The method comprises placing the patient on the insulated chair, attaching the conductive surface and nebulizer to the patient, charging the patient and nebulizer to a charge using the charging machine, providing aerosolized fluid to the patient using the nebulizer, then charging the patient to a charge opposite that of the nebulizer using the charging machine.

### FIELD OF THE INVENTION

The present invention relates generally to methods and apparatuses for delivery of particles to the body and specifically delivery of charged particles to the alveolus and small airways.

### BACKGROUND OF THE INVENTION

Many medicines are delivered into the bloodstream directly through an intravenous (IV) line in a patient's vein. The common and basic use of an IV is a needle piercing a the skin and entering a vein with a syringe, tubing, and/or IV bag attached. Other administration of medicine is in the form of pills that are taken orally and absorbed into the blood through the stomach walls. Each of these methods have their benefits and drawbacks. An IV line generally requires use of a needle and a medical professional to oversee the process. Further, people with certain conditions may be unable to take medicine intravenously, and even those who can take an IV may have a fear of needles that prevents them from using an IV. Alternatively, medication in pill form is easy to take and does not require breaking the skin or oversight from a medical professional, but absorption of the medication into the bloodstream is limited when compared to the direct approach of an IV line.

One means of medicine delivery that blends the ease and effectiveness of IVs and pills is delivery of aerosolized medication through the respiratory system. Commonly, a nebulizer is used to aerosolize a medication so that it can be breathed in by a patient and absorbed into the bloodstream at the patient's alveoli. Specifically, nebulizers turn a liquid medication into an inhalable aerosol that is easily breathed in by a patient, being quickly absorbed onto the patient's airways with less than five percent inhaled aerosol reaching the alveolus. Due to the physiological differences, the lungs act as a faster route of than the stomach. The alveolar-vascular barrier is thin and has a large surface area, making it an ideal location for fast absorption into the bloodstream.

Particles in an aerosol may be positively or negatively charged. These charges may occur naturally, or a charge can be actively put on particles by adding or stripping electrons. Similarly, a human body can become charged either through natural or purposeful processes. Charge affects particles based on their mass, meaning that a small particle would be noticeably affected by other charged particles, but a large body would be minimally affected by a small, charged particle. When a human body becomes charged, the effect is often unnoticeable because the charge is small compared to the size of the person. However, that charge can significantly affect particles in and around the body since the charge of the body may be significant to the small particles.

Charged aerosol particles inhaled by a person behave differently depending on the charge associated with the aerosol particle and the charge associated with the person. When a person and an aerosol have the same charge, the aerosol will be repelled from internal surfaces of the person's body such as the airways and the alveoli. Alternatively, when a person and an aerosol have opposite charges, the aerosol particles will be attracted to internal surfaces of the person's body such as the airways and alveoli. Thus, an aerosol with the same charge as a person will easily reach the lungs when inhaled because it won't be attracted to the walls of the airway, however, an aerosol with such a charge will also be repelled from the person's alveolar walls, where aerosolized medications can enter the bloodstream. Placing an opposite charge on a person once charged particles are already present in the person's lungs causes those particles to be attracted to the alveolar wall, leading to efficient absorption of the aerosol particles into the bloodstream. Thus, the charge of a person throughout inhalation of a charged aerosol can greatly affect the probability of the aerosol reaching the alveolus for rapid absorption.

Modern nebulizers can create particles smaller than one micron. However, even these submicron airway particles can be captured by respiratory cilia in the airway. Manipulating the charge of the airway, cilia, and alveolus in a person's body can thus allow the travel of aerosolized medications to the alveoli to be improved.

The present invention describes a system and method for more effectively delivering ionized particles to the bloodstream by taking advantage of the interactions between ionized particles and other charged objects.

### SUMMARY OF THE INVENTION

The present invention is a system and method for delivering charged particles. The system of the present invention comprises an electrical conducting medium, a conductive surface, a nebulizer, an insulated chair, and a charging machine. The charging machine is configured to provide an electrical charge to the electrical conducting medium by way of the conductive surface and to the nebulizer. The nebulizer is configured to aerosolize fluid and provide a flow of aerosolized fluid to the electrical conducting medium. The insulated chair is configured to keep the electrical conducting medium insulated from the electrical ground. The method of the present invention comprises placing the electrical conducting medium on the insulated chair, attaching the conductive surface and nebulizer to the electrical conducting medium, charging the electrical conducting medium and the nebulizer to the same charge using the charging machine, providing aerosolized fluid to the electrical conducting medium using the nebulizer, then charging the electrical conducting medium to a charge different from that of the nebulizer using the charging machine.

### BRIEF DESCRIPTION OF THE DRAWINGS

FIG. 1 is a diagram of the system of the present invention in accordance with at least one embodiment.

FIG. 2 is a diagram of the system of the present invention in accordance with at least one embodiment.

FIG. 3 is an illustration of the nebulizer of the present invention in accordance with at least one embodiment.

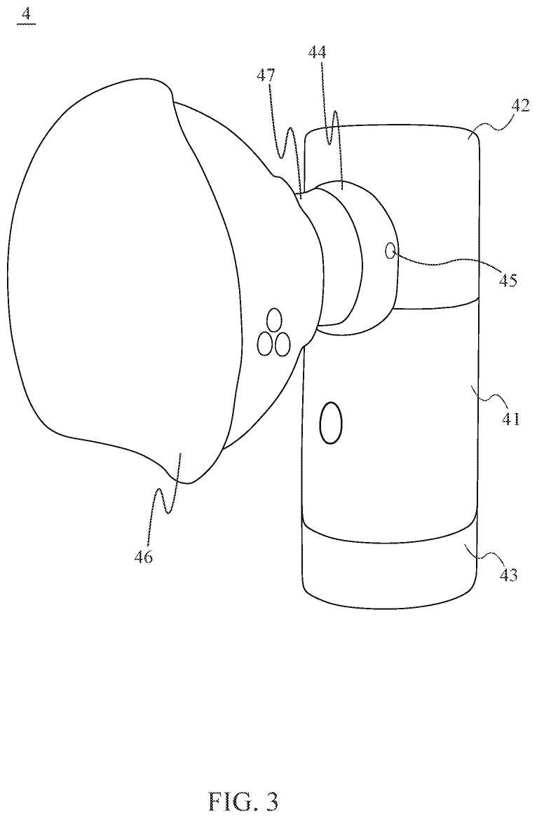

FIG. 4 is an illustration of the insulating chair of the present invention in accordance with at least one embodiment.

FIG. 5 is an illustration of the conductive surface of the present invention in accordance with at least one embodiment.

FIG. 6 is an illustration of the conductive surface of the present invention in accordance with at least one embodiment.

FIG. 7 is a flowchart of the method of the present invention in accordance with at least one embodiment.

FIG. 8 is a flowchart of the method of the present invention in accordance with at least one embodiment.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.” All illustrations of the drawings are for the purpose of describing selected versions of the present invention and are not intended to limit the scope of the present invention.

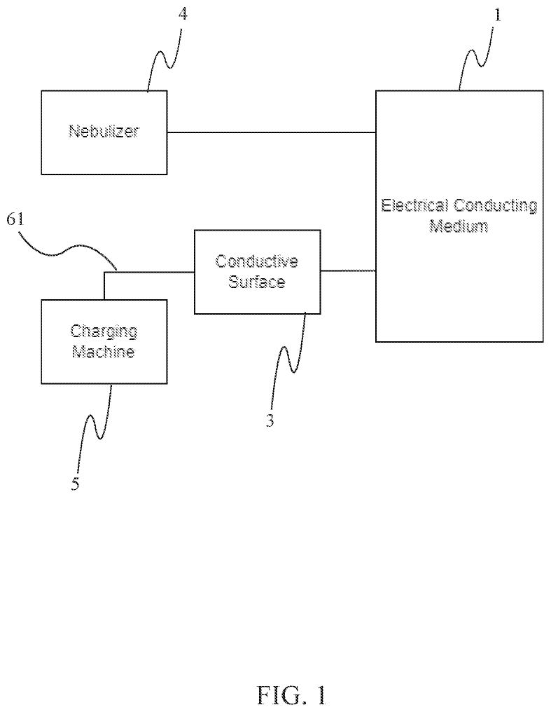

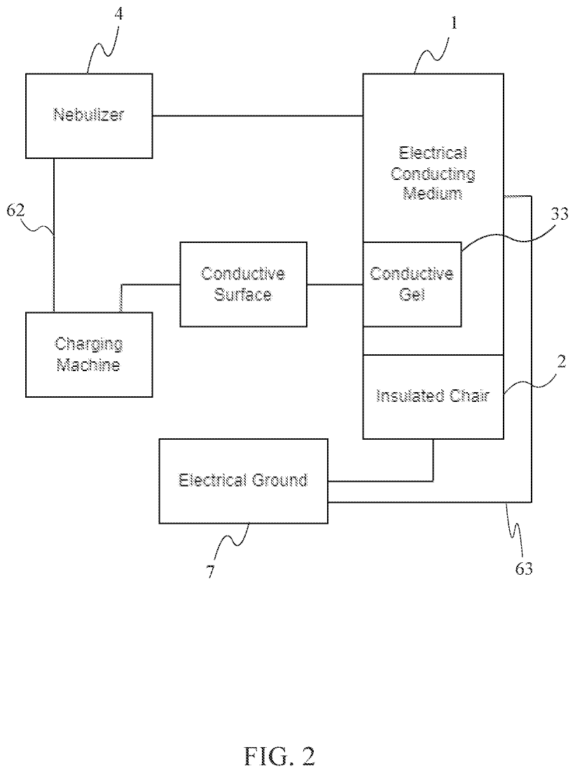

The present invention is a system and method for delivery of ionized aerosols to the alveoli in the human body. The system of the present invention, as shown in FIG. 1-2 and further described herein, comprises an electrical conducting medium 1, an insulated chair 2, a conductive surface 3, a nebulizer 4, and a charging machine 5. The method of the present invention comprises attaching each of the components of the system to a patient, charging the patient with the charging machine 5, charging medication to the same charge as the patient, providing charged aerosolized medication to the patient, then charging the patient to the opposite charge as the aerosolized medication. The system and method of the present application are designed to safely, easily, and effectively provide medication to a patient.

The electrical conducting medium 1 may be any object that conducts electricity and is capable of taking in aerosolized particles. In the preferred embodiment, the electrical conducting medium 1 is a person.

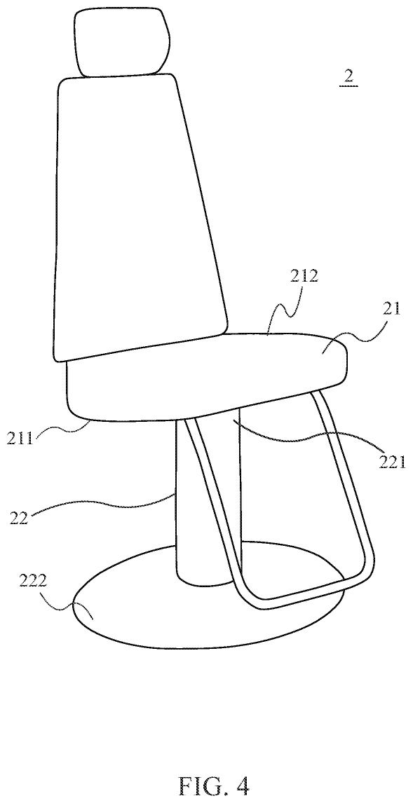

As shown in FIG. 4, the insulated chair 2 of the present invention comprises a seat 21 and an insulated stand 22. The seat 21 is any surface that a person or object could rest upon, having a base 211 and a seating surface 212. The insulated stand 22 is a platform having a first end 221 and a second end 222, the first end 221 connecting to the base 211 of the seat 21 and the second end 222 resting on the ground to keep the seat 21 elevated off the ground. The insulated stand 22 is made at least partially of an insulating material so any person or object resting on the seating surface 212 is electrically insulated from the ground. Such materials may be wood, rubber, glass, or any other material that insulate electricity. In the preferred embodiment, the insulated chair 2 of the present invention is designed much like a dental chair, having a large adjustable chair elevated off the ground by a single support beam. In alternative embodiments, insulated chair 2 may be designed much like a stool or a folding chair, with the insulated stand 22 being made up of a plurality of insulated legs.

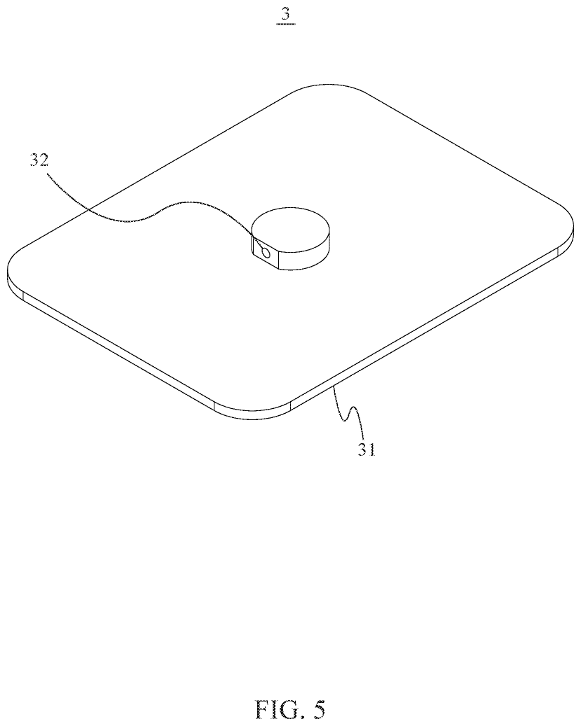

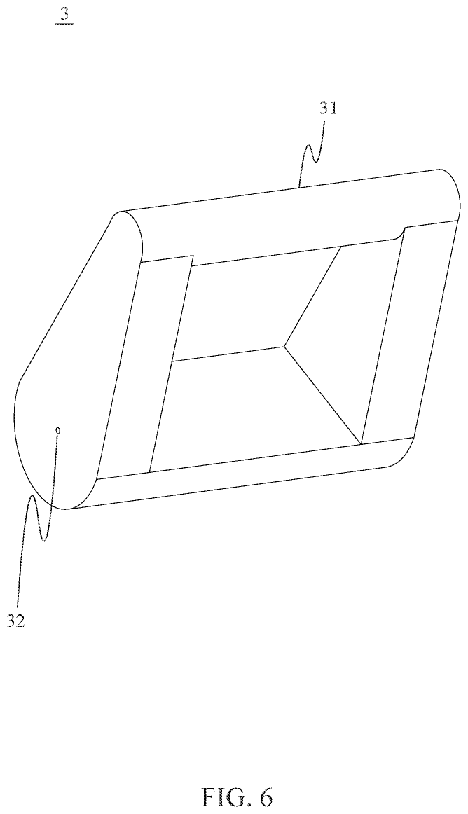

The conductive surface 3 of the present invention comprises a conductive face 31 and a port 32. The conductive face 31 may be any shape that allows contact to be made over a substantial surface area. The conductive face 31 is made of a conducting material so anything in contact with the conductive face 31 can be electrically connected to other components. The port 32 of the conductive surface 3 is connected to the conductive face 31 and configured to receive a charging wire 61. The charging wire 61 has a first end and a second end and is configured to electrically connect the conductive face 31 to another object. In the preferred embodiment shown in FIG. 5, the first end of the charging wire 61 is connected to the port 32 of the conductive surface 3. In one embodiment, the conductive surface 3 is a flat patch that can be adhered to a person or object. In another embodiment shown in FIG. 6, the conductive surface 3 is a cylindrical handle that can be grabbed by a person or connected to an object. The conductive surface 3 in conjunction with the electrical connector is configured to electrically connect two objects by placing one object against the conductive face 31 and touching the second end of the charging wire 61 to the second object.

The nebulizer 4 of the present invention comprises a nebulizer body 41, a medicine bowl 42, a compressor 43, an outlet 44, a charging port 45, a mouthpiece 46, and a flow meter 47. The compressor 43 and medicine bowl 42 are housed within the nebulizer body 41. The outlet 44 is attached to the nebulizer body 41 near the medicine bowl 42 and connects the mouthpiece 46 to the nebulizer body 41 and medicine bowl 42. The flow meter 47 is attached to the mouthpiece 46 and configured to measure the rate of flow of aerosolized fluid through the mouthpiece 46. The flow meter 47 may be any device capable of measuring the mass flow rate of gas, such as a turbine flow meter, or thermal mass flow meter. The charging port 45 may be placed at any point on the nebulizer body 41 or the outlet 44, but in the preferred embodiment, the charging port 45 is an opening placed near where the outlet 44 meets the nebulizer body 41. The placement of the charging port 45 allows an electric charge to be placed on the aerosolized fluid at or shortly after the point of aerosolization. The charging port 45 is configured to receive a wire or other means of introducing electric charge into the nebulizer 4. In use, the medicine bowl 42 is filled with a fluid and the compressor 43 is activated, causing the liquid to aerosolize and be expelled through the outlet 44 and the mouthpiece 46. In the preferred embodiment, the charging port 45 is connected to the nebulizer body 41 is connected to the nebulizer body 41 near the outlet 44, in order to introduce electric charge to the liquid as it is in the process of aerosolizing.

The charging machine 5 of the present invention comprises a machine body 51, a charge creation mechanism 52, a plurality of ports 53, a grounding wire 63, and a nebulizer wire 62. The machine body 51 of the charging machine 5 houses the charge creation mechanism 52. The charge creation mechanism 52, in the preferred embodiment, is an electrostatic generator configured to accumulate electric charge, creating a high voltage direct current electricity at low current levels. In some embodiments, the charge creation mechanism 52 may operate by friction or by electrostatic induction to convert work into electrostatic energy. The voltage and amperage of the direct current electricity may be predetermined by its operator. The charge creation mechanism 52 may produce an electric potential of hundreds of kilovolts, while the current is generally kept below ten milliamperes as not to cause pain or injury to a person put in electrical contact with the charge creation mechanism 52. The charging machine 5 is configured so the electrical potential produced by the charge creation mechanism 52 can place either a positive or negative charge on any electrically connected components. Other components may be connected to the charging machine 5 by the plurality of ports 53 connected to the machine body 51 and configured to accept wires. The plurality of ports 53 comprise at least a first port 531 connected to the charge creation mechanism 52, a second port 532 connected to the charge creation mechanism 52, and a third port 533 connected to an electrical ground 7. The nebulizer wire 62 is connected to the second port 532 and the grounding wire 63 is connected to the third port 533. The grounding wire 63 may additionally comprise a resistor 631 to resist the flow of electricity through the grounding wire 63. The charging machine 5 in the preferred embodiment is configured to produce a first charge and a second charge, each charge simultaneously created and transmittable to the other components via wires. In the preferred embodiment, the first charge and the second charge are oppositely charged.

The system of the present invention, as shown in FIG. 1-2, is configured as follows. The electrical conducting medium 1 is placed atop the insulating chair on the seating surface 212 of the seat 21. In the preferred embodiment, the electrical conducting medium 1 is a patient who is seated on the seating surface 212. The conductive surface 3 is connected to the electrical conducting medium 1 by placing any part of the electrical conducting medium 1 in contact with the conductive face 31. In some embodiments, an adhesive material or strap is used to fasten the conductive surface 3 to the electrical conducting medium 1 and a conductive gel 33 may be applied to the electrical conducting medium 1 to promote electrical conductivity between the conductive surface 3 and the electrical conducting medium 1. In embodiments in which the electrical conducting medium 1 is a patient, the electrical conducting medium 1 is connected to the conductive surface 3 by placing the patient's hand on the conductive face 31 or attaching the conductive face 31 to the patient's chest, as is commonly done with a defibrillator. The nebulizer 4 is attached to the electrical conducting medium 1 by the mouthpiece 46 of the nebulizer 4 so aerosolized fluid that flows out of the mouthpiece 46 flows directly to the electrical conducting medium 1. In the preferred embodiment in which the electrical conducting medium 1 is a human patient, the mouthpiece 46 is a mask that is placed over the patient's nose and mouth so aerosolized fluid from the nebulizer may be breathed in by the patient. The charging machine 5 is connected to the conductive surface 3 by the charging wire 61, the charging wire 61 connecting to the first port 531 of the charging machine 5 and the port 32 of the conductive surface 3. This connection allows the charge created by the charge creation mechanism 52 of the charging machine 5 to be placed on the conducting surface and subsequently the electrical conducting medium 1. The charging machine 5 is also connected to the nebulizer 4 by the nebulizer wire 62, the nebulizer 4 wire being connected to the second port 532 of the charging machine 5 and the charging port 45 of the nebulizer 4. This connection allows the charge created by the charge creation mechanism 52 of the charging machine 5 to be placed on the outlet 44 and/or medicine bowl 42 of the nebulizer 4.

In embodiments which comprise a flow meter 47, the flow meter 47 is attached to the outlet 44 or mouthpiece 46 of the nebulizer 4 and is configured to measure the rate of flow of aerosolized fluid from the nebulizer 4 to the electrical conducting medium 1. The flow meter 47 may be communicatively connected to the charging machine 5, either wired or wirelessly. The flow meter 47 may be configured to communicate the flow rate of aerosolized fluid to the charging machine 5. In such embodiments, the charging machine 5 may be configured to adjust the charge on the conductive surface 3 based upon the flow rate of aerosolized fluid. In one embodiment, the charging machine 5 may provide a first charge to the conductive surface 3 when a first flow rate is measured by the flow meter 47, then the charging machine 5 may provide a second charge to the conductive surface 3 when a second flow rate is measured by the flow meter 47. For example, in an embodiment where the electrical conducting medium 1 is a human patient, the charging machine 5 may provide a positive charge to the conductive surface 3 when the flow rate is above a predetermined rate due to inhalation by the patient. Once the patient seizes inhalation, the charging machine 5 may then provide a negative charge to the conductive surface 3 due to the decreased flow rate. This example exemplifies the method and system in which an aerosolized fluid of the same charge as the patient is breathed in, then the patient is changed to a charge opposite the aerosolized fluid in order to promote delivery of the charged aerosolized fluid to the patient's airways, and eventually bloodstream. While the flow rate in this example is based on inhalation by the patient, other embodiments allow the flow rate of aerosolized fluid from the nebulizer 4 to be manually controlled or regularly programmed.

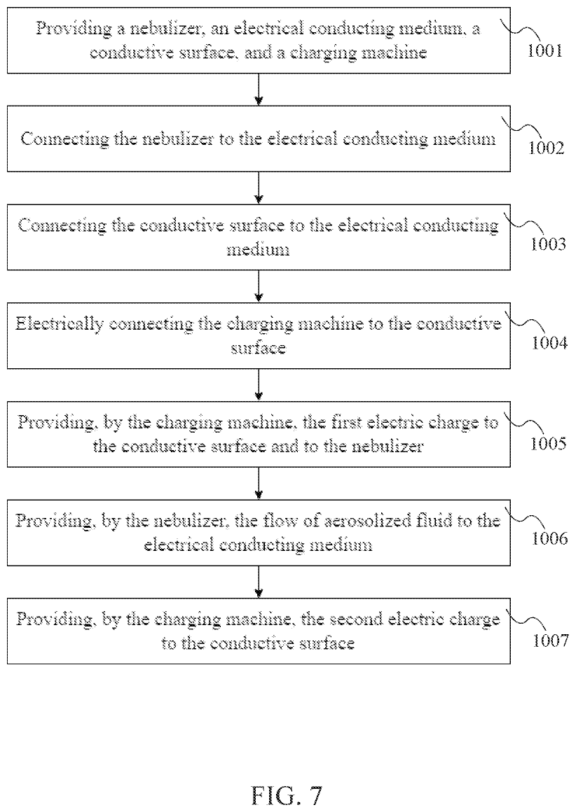

The method of the present invention and its alternative embodiments, as shown in FIG. 7, are as follows. The first step of the method is providing a nebulizer, an electrical conducting medium, a conductive surface, and a charging machine 1001. These apparatuses may include any of the subcomponents and details of the above-described system. Particularly, the provided nebulizer must be configured to provide a flow of aerosolized fluid and the provided charging machine must be configured to produce a first electric charge and a second electric charge and provide the first electric charge and the second electric charge to the conductive surface and the nebulizer. The next steps of the method are connecting the nebulizer to the electrical conducting medium 1002, connecting the conductive surface to the electrical conducting medium 1003, and electrically connecting the charging machine to the conductive surface 1004. The charging machine then provides the first electric charge to the conductive surface and to the nebulizer 1005. The nebulizer then provides the flow of aerosolized fluid to the electrical conducting medium 1006. Finally, the charging machine provides the second electric charge to the conductive surface 1007.

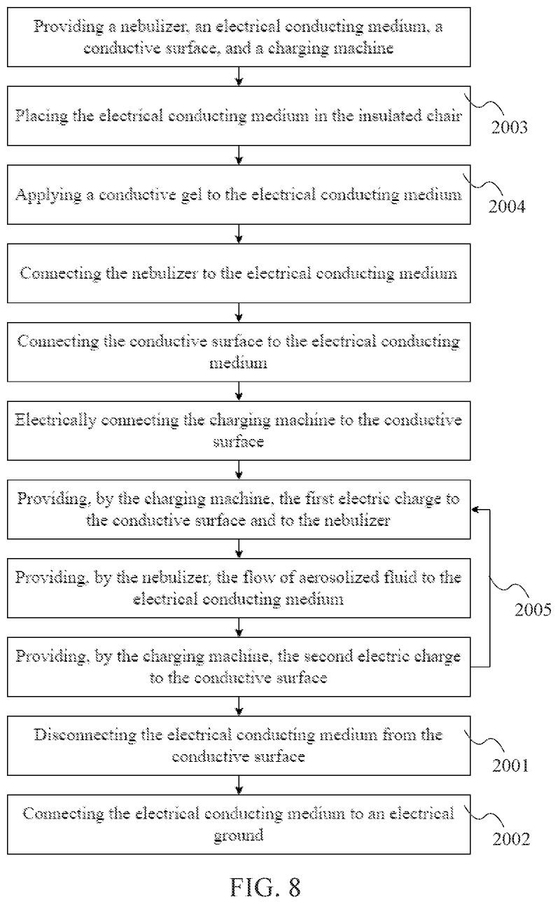

Additional steps present in alternative embodiments, as shown in FIG. 8, may be used to carry out the method of the present invention in a more specific manner. An embodiment may include providing a grounding wire, disconnecting the electrical conducting medium from the conductive surface 2001, and connecting the electrical conducting medium to an electrical ground by the grounding wire 2002. Another embodiment may include providing a resistor and placing it between the grounding wire and the electrical conducting medium to connect the electrical conducting medium to the electrical ground 2002. The method may also include the steps of providing an insulated chair having a seat and an insulated stand 2003, configured to keep the seat electrically disconnected from the electrical ground, and placing the electrical conducting medium on the seat of the insulated chair. Embodiments of the method may also include the applying a conductive gel to the electrical conductive medium prior to connecting the electrical conductive medium to the conductive surface 2004.

In addition to the above-mentioned alterations to the method of the present invention, the method of the present invention may include repeating any sequence of steps a predetermined number of times at any predetermined interval. For example, the steps of providing the first electric charge through providing the second electric charge may be repeated as necessary 2005. The method may also incorporate additional components, subcomponents, and configurations of any of the components discussed in the context of the system of the present invention.

Although the invention has been explained in relation to its preferred embodiment, it is to be understood that many other possible modifications and variations can be made without departing from the spirit and scope of the invention.

What is claimed is:

1. A system for delivery of charged particles, comprising:
a nebulizer;
an electrical conducting medium;
a conductive surface;
a charging machine;
the nebulizer further comprising a nebulizer body, a medicine bowl, a compressor, an outlet, and a mouthpiece;
the nebulizer being configured to aerosolize a fluid placed in the medicine bowl and expel the aerosolized fluid through the outlet and the mouthpiece;
the nebulizer being attached to the electrical conducting medium;
the conductive surface being attached to the electrical conducting medium;
the conductive surface being attached to the charging machine by a charging wire; and
the charging machine being configured to produce a first electric charge and provide the first electric charge to the conductive surface.

2. The system for delivery of charged particles of claim 1, further comprising:
an insulated chair;
the insulated chair further comprising a seat and an insulated stand;
the insulated stand having a first end and a second end;
the first end of the insulated stand connecting to the seat;
the insulated chair configured to keep the seat electrically disconnected from the second end of the insulated stand; and
the electrical conducting medium being connected to the seat.

3. The system for delivery of charged particles of claim 2, further comprising:
a nebulizer wire;
the nebulizer further comprising a charging port;
the charging machine being connected to the nebulizer by the nebulizer wire; and
the charging machine being configured to provide an electric charge to the charging port of the nebulizer.

4. The system for delivery of charged particles of claim 3, further comprising:
a grounding wire;
an electrical ground; and
the electrical conducting medium being electrically connected to the electrical ground by the grounding wire.

5. The system for delivery of charged particles of claim 3, further comprising:
a flow meter;
the flow meter connected to the outlet of the nebulizer; and
the flow meter configured to measure a flow rate through the outlet of the nebulizer.

6. The system for delivery of charged particles of claim 5, further comprising:
the flow meter configured to communicate the flow rate to the charging machine; and
the charging machine configured to produce a second electric charge and provide the second electric charge to the conductive surface.

7. The system for delivery of charged particles of claim 3, further comprising:
a conductive gel; and
the conductive gel covering the electrical conducting medium.

8. The system for delivery of charged particles of claim 3, further comprising:
the conductive surface being an adhesive patch having a flat conductive face and a port;
the conductive surface connecting to the electrical conducting medium at the conductive face; and
the charging wire connecting to the conductive surface at the port.

9. The system for delivery of charged particles of claim 3, further comprising:
the conductive surface being a handle having a cylindrical conductive face and a port;
the conductive surface connecting to the electrical conducting medium at the conductive face; and
the charging wire connecting to the conductive surface at the port.

10. The system for delivery of charged particles of claim 3, further comprising:
the charging machine being an electrostatic generator configured to accumulate electric charge and create a direct current electricity with a predetermined voltage and a predetermined amperage.

11. The system for delivery of charged particles of claim 3, further comprising:
the predetermined voltage being greater than 10 kilovolts; and
the predetermined amperage being less than 10 milliamperes.

12. A method for delivery of charged particles, comprising:
providing a nebulizer, an electrical conducting medium, a conductive surface, and a charging machine;
the nebulizer being configured to provide a flow of aerosolized fluid;
the charging machine being configured to produce a first electric charge and a second electric charge and provide the first electric charge and the second electric charge to the conductive surface and the nebulizer;
connecting the nebulizer to the electrical conducting medium;
connecting the conductive surface to the electrical conducting medium;
electrically connecting the charging machine to the conductive surface;
providing, by the charging machine, the first electric charge to the conductive surface and the nebulizer;
providing, by the nebulizer, the flow of aerosolized fluid to the electrical conducting medium; and
providing, by the charging machine, the second electric charge to the conductive surface.

13. The method for delivery of charged particles of claim 12, further comprising:
providing a grounding wire;
disconnecting the electrical conducting medium from the conductive surface; and
connecting the electrical conducting medium to an electrical ground by the grounding wire.

14. The method for delivery of charged particles of claim 13, further comprising:
providing a resistor;
the step of connecting the electrical conducting medium to an electrical ground further comprising placing the resistor between the grounding wire and the electrical conducting medium.

15. The method for delivery of charged particles of claim 12, further comprising:
providing an insulated chair having a seat and an insulated stand;
the insulated chair being configured to keep the seat electrically disconnected from an electrical ground; and
placing the electrical conductive medium on the seat of the insulated chair.

16. The method for delivery of charged particles of claim 12, further comprising:
repeating the steps of providing the first electric charge through providing the second electric charge a predetermined number of times.

17. The method for delivery of charged particles of claim 12, further comprising:
the nebulizer further comprising a flow meter; and
the flow meter being configured to communicate a flow rate of aerosolized fluid to the charging machine.

18. The method for delivery of charged particles of claim 12, further comprising:
applying a conductive gel to the electrical conducting medium before the step of connecting the conductive surface to the electrical conducting medium.

19. The method for delivery of charged particles of claim 12, further comprising:
the charging machine being an electrostatic generator configured to accumulate electric charge and create a direct current electricity with a predetermined voltage and a predetermined amperage.

20. The method for delivery of charged particles of claim 12, further comprising:
the predetermined voltage being greater than 10 kilovolts; and
the predetermined amperage being less than 10 milliamperes.
